(19) United States
(12) Patent Application Publication
(10) Pub. No.: US 2007/0059824 A1
(43) Pub. Date: Mar. 15, 2007

(54) Human umbilical cord blood-derived pluripotent fibroblast-like-macrophages

(76) Inventors: Yong Zhao, Lisle, IL (US); Theodore Mazzone, Wilmette, IL (US)

Correspondence Address: TODD S. PARKHURST;HOLLAND & KNIGHT LLP, 131 S. DEARBORN STREET, 30TH FLOOR, CHICAGO, IL, 60603, US

(21) Appl. No.: 11/519,307

(22) Filed: Sep. 12, 2006

Related U.S. Application Data
(60) Provisional application No. 60/716,261, filed on Sep. 12, 2005.

(51) Int. Cl. C12N 5/08

(52) U.S. Cl. 435/372

(57) ABSTRACT

The present invention relates to a purified population of fibroblast-like macrophage (f-macrophage, f-MΦ) and methods using the same. The f-MΦ can be expanded in vitro and differentiated into several lineages, including insulin-expressing cells, endothelial cells, and neuronal cells. The f-MΦ described herein have been generated from human umbilical cord blood (CB f-MΦ) and have characteristics similar to f-MΦ derived from peripheral blood. Thrombopoietin (TPO), at low dosage, significantly stimulates the proliferation of CB f-MΦ, wherein the TPO-expanded CB f-MΦ retain their pluripotent differentiation potential.

This application claims priority benefit from U.S. provisional application Ser. No. 60/716,261 filed Sep. 12, 2005.

### TECHNICAL FIELD

The present invention relates to a purified population of fibroblast-like macrophage (f-macrophage, f-MΦ and methods using the same. The f-MΦ can be expanded in vitro and differentiated into several lineages, including insulin-expressing cells, endothelial cells, and neuronal cells. The f-MΦ described herein have been generated from human umbilical cord blood (CB f-MΦ) and have characteristics similar to f-MΦ derived from peripheral blood. Thrombopoietin (TPO), at low dosage, significantly stimulates the proliferation of CB f-MΦ, wherein the TPO-expanded CB f-MΦ retain their pluripotent differentiation potential.

### BACKGROUND OF THE INVENTION

Advances in stem cell biology, including embryonic and adult stem cells, have provided potential tools for cell replacement therapy for treating serious human diseases such as cancer, genetic diseases, diabetes, neuronal disease, and cardiovascular disease. However, a shortage of suitable donors, immune rejection, and ethical issues significantly limit these approaches for broad application. Heterogeneous transplantation of mature cells as replacement therapy is limited by the availability of donors and need for long-term immunosuppressive drugs. Recently, autologous stem cells have been found in many adult tissues such as brain, heart, and pancreas, in addition to bone marrow. The major limitation to the therapeutic use of these cells is their relatively low cell number and their reduced potential for proliferation. To immortalize such cells, the use of cell feeders and/or growth factor genes to expand stem cell availability raises potential problems with contamination of cell feeders and tumorigenicity. Therefore, new strategies are needed before adult stem cells can become a practical therapeutic approach.

To date, stem cells found in bone marrow and umbilical cord blood have been used extensively to repopulate the hematopoietic system. The use of cord blood has several unique advantages, including no risk to the donor, low risk of graft-versus-host disease, and rapid availability. The major disadvantage of cord blood transplantation, however, is the low number of hematopoietic progenitor cells (CD34⁺ cells) compared with bone marrow or mobilized peripheral blood.

An object of the present invention is to provide purified populations of cord blood derived stem cells, for example f-MΦ, which can be expanded in vitro and differentiated into several lineages, including insulin-expressing cells, neuronal cells, and endothelial cells.

### SUMMARY OF THE INVENTION

Using cord blood as an alternative source of stem cells has been performed for the treatment of cancer and genetic diseases. The CD34⁺ subpopulation of human umbilical cord blood is generally sorted and used for transplantation. However, low yields of CD34⁺ cells in cord blood limit their practical application. See, for example, K. K. Ballen, Blood, 105 (2005) 2786-3792. The present invention is directed to an alternative subpopulation to generate stem cell from cord blood. As described herein, cord blood-derived monocytes were treated with M-CSF thereby generating a pluripotent stem cell, CB f-MΦ. The CB f-MΦ displayed characteristics similar to that of the peripheral blood-derived f-MΦ (see Zhao et al., Proc. Natl. Acad. Sci. USA 100 (2003) 2426-2431); including hematopoietic stem cell marker CD34, along with expressing macrophage markers CD14, CD163, and phagocytosis, leukocyte common antigen CD45. This type of stem cell has not been previously recognized in cord blood.

A major limitation to primary stem cell-based therapy is the need to generate sufficient numbers of cells retaining their pluripotentiality. Cell number can be increased by introduction of growth stimulatory genes to produce sustained expansion. However, loss of differentiation potential and safety concerns has limited the usefulness of this approach. In addition, promoting cell growth in an undifferentiated state by co-incubation with feeder layers increases the risk for cross-transfer of pathogens. Therefore, the use purified recombinant growth factors has been widely applied to expand stem cell cultures. Thrombopoietin (TPO) has been shown to have multiple functions not only inducing differentiation to a platelet and megakaryocytic phenotype, but also stimulating the proliferation of hematopoietic cells (See B. Fishley, W. S. Alexander, Thrombopoietin signalling in physiology and disease, Growth Factors 22 (2004) 151-155; and D. J. Kuter, C. G. Begley, Recombinant human thrombopoietin: basic biology and evaluation of clinical studies, Blood 100 (2002) 3457-3469). The present invention demonstrates that TPO, at low dosage, can stimulate CB f-MΦ proliferation without altering their phenotype. The herein described data also confirm that megakaryocytic differentiation is not induced. The below-described examples will illustrate the present invention by showing that CB f-MΦ retain their pluripotent ability and can give rise to the endothelial-like and insulin-expressing cells after TPO expansion, Hence, the large-scale cultivation of CB f-MΦ through TPO treatment is a therapeutic approach for obtaining a sufficient number of adult stem cell for practical application.

The present invention is directed to purified populations of mammalian cord blood derived f-MΦ. Using human umbilical cord blood as a novel source, methods of isolating and purifying cord blood-derived f-MΦ (CB fMΦ) are disclosed. The CB f-MΦ of the present invention are generally derived from umbilical cord blood-derived monocytes and have characteristics similar to the peripheral blood-derived f-MΦ. CB f-MΦ is easy to obtain using M-CSF treatment and be amplified by TPO stimulation. In addition, unlike the bone marrow aspiration and drop blood, there are no risks to donors and no opportunity infection using cord blood to generate CB f-MΦ. CB f-MΦ of the present invention can be induced to proliferate in vitro with the administration of low doses of thrombopoietin (TPO). The TPO-expanded CB f-MΦ retain their pluripotent differentiation potential.

The present invention provides pharmaceutical compositions including one or more of f-MΦ, methods of preparing and sustaining the one or more f-MΦ, methods of propagating one or more f-MΦ, methods of differentiating one or more f-MΦ, and methods of using one or more f-MΦ to treat diseases or to ameliorate symptoms associated with a disease or disorder. The CB f-MΦ of the present invention are derived from cord blood monocytes.

In one embodiment, the present invention provides a method of preparing an isolated monocyte-derived stem cell comprising the steps of isolating a cord-blood monocyte; contacting the cord blood monocyte with an effective amount of a mitogenic compound selected from the group consisting of macrophage colony-stimulating factor (M-CSF), interleukin-6, human recombinant interleukin-12, β-nerve growth factor, vascular endothelial growth factor isoform 165, hepatocyte growth factor (HGF), basic fibroblast growth factor (bFGF), phorbol 12-myristate 13-acetate (PMA), lipopolysaccharide (LPS), and leukemia inhibitory factor (LIF); and culturing the cord-blood monocyte under conditions suitable for propagation of the monocyte and thereby obtaining a preparation of an isolated cord-blood monocyte-derived stem cell. The cord blood monocyte is preferably a mammalian, or human cord blood monocyte. In one embodiment, the cord blood monocyte is cryopreserved prior to contact with a mitogenic compound. In a related aspect of the invention, the isolated cord-blood monocyte derived stem cell is cryopreserved.

In another embodiment, the present invention comprehends an isolated cord-blood derived stem cell obtained by the foregoing described method. As the cord-blood derived stem cell of the present invention has a distinct phenotype, it is contemplated that the cord-blood derived stem cell will have at least one specific and characteristic activity. For example, a cord-blood derived stem cell of the present invention exhibits at least one distinct cell surface marker or produces at least one cytokine selected from the group consisting of IL-1β, IL-6, and IL-12 p70, or exhibits phagocytic activity, or exhibits lymphocyte activation activity, or exhibits resistance to dispersion by any one of trypsin, EDTA and dispase, or exhibits susceptibility to dispersion by lidocaine. Examples of such surface markers include: CD14, CD45, CD34, CD117, CD163, CD40, and MAC-1. Preferably, an isolated cord blood-derived stem cell of the present invention exhibits phagocytic activity. Also preferred is an isolated cord blood-derived stem cell exhibiting at least one of the above-identified cell surface markers, production of one of the above-identified cytokines, phagocytic activity, lymphocyte activation activity, resistance to dispersion by trypsin, EDTA, or dispase, and susceptibility to dispersion by lidocaine.

Isolated cord blood-derived stem cells exhibiting a variety of cell-surface antigens are contemplated in the invention. In one embodiment of the present invention, an isolated cord blood-derived stem cell is provided wherein the cell exhibits a surface antigen selected from the group consisting of CD14, CD45, CD34, CD117, CD163, CD40, and MAC-1. In another embodiment, the invention provides an isolated cord blood-derived stem cell that exhibits phagocytic activity.

In another embodiment of the present invention, a method of generating a differentiated cell is provided comprising the steps of isolating a cord blood derived stem cell and contacting the cell with an amount of an inducing agent effective to induce differentiation of the cell. Preferably, the differentiated cell is cultured under conditions for sustaining and/or propagating the cell. The cord blood derived stem cell of the present invention is preferably a human cord blood derived stem cell. In a related embodiment, the present invention contemplates cryopreservation of the cord blood derived stem cell and/or the differentiated cell.

In yet another embodiment of the present invention, a method of identifying cell type specific therapeutic agents is provided. The method comprises contacting a candidate therapeutic agent and a first differentiated cell obtained according to the above-described method of generating a differentiated cell, further contacting the candidate therapeutic agent and second differentiated cell obtained according to that method of generating a differentiated cell, wherein the first and second differentiated cells are different cell types, and measuring the viability of the first differentiated cell relative to the viability of the second differentiated cell, wherein a difference in viabilities identifies the candidate therapeutic agent as a cell type specific therapeutic agent.

Give the scope of the invention, one skilled in the art will appreciate that a variety of growth and differentiation factors and methods, which are employed in the generation, sustaining and/or propagation of a range of specific cell and tissue types, can be used in sustaining, propagation and/or differentiation of the cord blood derived stem cell described herein. In particular, the invention contemplates a method of generating, sustaining and/or propagating an endothelial cell; wherein the method comprises the steps of isolating a cord blood derived stem cell; contacting the cord blood derived stem cell with an amount of an endothelial cell inducing agent such as vascular endothelial growth factor (VEGF) effective to induce cord blood derived stem cell differentiation into an endothelial cell; and culturing the endothelial cell under conditions suitable for sustaining and/or propagating the endothelial cell. Optionally, and prior to contacting the cord blood derived stem cell with an inducing agent, the cord blood monocyte derived stem cells may be expanded via treatment with an effective amount of thrombopoietin.

In another aspect of the invention, a method of generating, sustaining and/or propagating an insulin-expressing cell is provided comprising the steps of isolating a cord blood derived stem cell; contacting the cord blood derived stem cell with an amount of an insulin-expressing cell inducing agent such as lipopolysaccharide in the presence of high glucose effective to induce cord blood derived stem cell differentiation into an insulin-expressing cell; and culturing the insulin expressing cell under conditions suitable for sustaining and/or propagating the insulin expressing cell. Optionally, and prior to contacting the cord blood derived stem cell with an inducing agent, the cord blood monocyte derived stem cells may be expanded via treatment with an effective amount of thrombopoietin.

In another aspect of the invention, a method of generating, sustaining and/or propagating an epithelial cell is provided comprising the steps of isolating a cord blood derived stem cell; contacting the cord blood derived stem cell with an amount of an epidermal cell inducing agent such as epidermal growth factor (EGF) effective to induce cord blood derived stem cell differentiation into an epithelial cell; and culturing the epithelial cell under conditions suitable for sustaining and/or propagating the epithelial cell. Optionally, and prior to contacting the cord blood derived stem cell with an inducing agent, the cord blood monocyte derived stem cells may be expanded via treatment with an effective amount of thrombopoietin.

In yet another aspect of the invention, a method of generating, sustaining and/or propagating a T-lymphocyte is provided comprising the steps of isolating a cord blood derived stem cell; contacting the cord blood derived stem cell with an amount of a T-cell inducing agent such as interleukin-2 (IL-2) effective to induce cord blood derived stem cell differentiation into a T-lymphocyte; and culturing the T-lymphocyte under conditions suitable for sustaining and/or propagating the T-lymphocyte. Optionally, and prior to contacting the cord blood derived stem cell with an inducing agent, the cord blood monocyte derived stem cells may be expanded via treatment with an effective amount of thrombopoietin.

In still another aspect of the invention, a method of generating, sustaining and/or propagating a macrophage is provided comprising the steps of isolating a cord blood derived stem cell; contacting the cord blood derived stem cell with an amount of a macrophage inducing agent such as lipopolysaccharide (LPS) effective to induce cord blood derived stem cell differentiation into a macrophage; and culturing the macrophage under conditions suitable for sustaining and/or propagating the macrophage. Optionally, and prior to contacting the cord blood derived stem cell with an inducing agent, the cord blood monocyte derived stem cells may be expanded via treatment with an effective amount of thrombopoietin.

In an additional aspect of the invention, a method of generating, sustaining and/or propagating a hepatocyte is provided comprising the steps of isolating a cord blood derived stem cell; contacting the cord blood derived stem cell with an amount of a hepatocyte inducing agent such as hepatocyte growth factor (HGF) effective to induce cord blood derived stem cell differentiation into a hepatocyte; and culturing the hepatocyte under conditions suitable for sustaining and/or propagating the hepatocyte. Optionally, and prior to contacting the cord blood derived stem cell with an inducing agent, the cord blood monocyte derived stem cells may be expanded via treatment with an effective amount of thrombopoietin.

In an additional aspect of the invention, a method of generating, sustaining and/or propagating a neuronal cell comprising the steps of isolating a cord blood derived stem cell; contacting the cord blood derived stem cell with an amount of a nerve cell inducing agent such as nerve growth factor (bNGF) effective to induce cord blood derived stem cell differentiation into a neuronal cell; and culturing the neuronal cell under conditions suitable for sustaining and/or propagating the neuronal cell. Optionally, and prior to contacting the cord blood derived stem cell with an inducing agent, the cord blood monocyte derived stem cells may be expanded via treatment with an effective amount of thrombopoietin.

In an additional aspect of the invention a method of generating, sustaining and/or propagating a retinal pigment epithelial cell comprising the steps of isolating a cord blood derived stem cell; contacting the cord blood derived stem cell with an amount of a retinal pigment epithelial cell inducing agent such as differentiating medium effective to induce cord blood derived stem cell differentiation into a retinal pigment epithelial cell; and culturing the retinal pigment epithelial cell under conditions suitable for sustaining and/or propagating the retinal pigment epithelial cell. Optionally, and prior to contacting the cord blood derived stem cell with an inducing agent, the cord blood monocyte derived stem cells may be expanded via treatment with an effective amount of thrombopoietin.

Preferably, a cord blood derived stem cell of the invention is isolated from a mammalian source. Also preferred are human sources for the cord blood derived stem cell according to the invention.

The use of isolated cord blood derived stem cells for the treatment of various diseases and disorders is further contemplated by the present invention. A disorder amenable to cell-based treatment includes, but is not limited to, Alzheimer's disease, Parkinson's disease, senile dementia, multiple sclerosis, age-related central nervous system (CNS) conditions, including changes manifested, e.g., as current time, date, location, or identity confusion, and/or recent memory loss, Acquired Immune Deficiency Syndrome (AIDS)-associated dementia, brain damage due to a blood clot, interruption of blood supply, formation or presence of a cyst, an autoimmune disorder, bacterial infection, e.g., of the brain, which may include an abscess, viral infection, e.g., of the brain, brain tumor, seizure disorders, neural trauma, surgical incision, diabetic ulcer, hemophiliac ulcer, varicose ulcer, solid angiogenic tumor, leukemia, hemangioma, acoustic neuroma, neurofibroma, trachoma, pyogenic granuloma, rheumatoid arthritis, psoriasis, diabetic retinopathy, retinopathy of premature macular degeneration, corneal graft rejection, neovascular glaucoma, retrolental fibroplasia, rubeosis, Osler-Webber Syndrome, myocardial angiogenesis blindness, plaque neovascularization, telangiectasia, hemophiliac joint, angiofibroma, wound granulation, epithelial cell neoplasia, Crohn's disease, chemical-, heat-, infection- or autoimmune-induced intestinal tract damage, chemical-, heat-, infection- or autoimmune-induced skin damage, systemic lupus erythematosus, AIDS, reactive arthritis, Lyme disease, insulin-dependent diabetes, an organ-specific autoimmune disorder, rheumatoid arthritis, inflammatory bowel disease, Hashimoto's thyroiditis, Grave's disease, contact dermatitis, psoriasis, graft rejection, graft-versus-host disease, sarcoidosis, a gastrointestinal allergy, eosinophilia, conjunctivitis, glomerular nephritis, a helminthic infection, lepromatous leprosy, diabetes, Gaucher's disease, Niemann-Pick disease, a parasitic infection, cancer, a disorder of the immune system, chemical (including drugs and alcohol)-, physical-, infection-, or autoimmune-induced hepatotoxicity, liver cancer, liver damage induced by metastatic cancer, a liver blood clot, and the degenerative diseases of the retina such as retinitis pigmentosa, and other retinal diseases related to the loss of retinal photoreceptor cells.

According to the present invention, the cord blood derived stem cell(s) is preferably isolated from the organism to receive treatment (i.e. is an autologous cord blood derived stem cell). Preferably, the cord blood derived stem cell(s) used to treat a disorder is derived from a mammalian, or human source.

It is contemplated that administration of one or more cell types according to the invention (e.g. cord blood derived stem cells and both non-terminally and terminally differentiated cells thereof) may be used to treat a disease or disorder or to ameliorate a symptom associated with such a disease or disorder.

Pharmaceutical compositions are also contemplated. Preferably, a pharmaceutical composition of the invention comprises a cord blood derived stem cell(s) and a pharmaceutically acceptable diluent, carrier or medium. The invention further contemplates a kit comprising a pharmaceutical composition according to the present invention.

Other features and advantages of the invention will be better understood upon review of the brief description of the drawings and the detailed description.

### FIGURES AND DRAWINGS

FIG. 1. Monocyte cell proliferation demonstrated by cell counting. White bars show the control monocytes cultured in the RPMI 1640 medium supplemented with 7% FBS without M-CSF; the black bar show the monocytes cultured in the same medium with 50 ng/ml M-CSF. Data represent one of at least five experiments with similar results.

FIG. 2. Effects of TPO on the CB f-MΦ. TPO stimulated the proliferation of the CB f-MΦ. Monocytes cultured in the RPMI 1640 medium supplemented with 7% FBS without M-CSF and TPO treatments served as control (dashed line). Data represent one of three independent experiments with the similar results.

FIG. 3. Insulin-expressing cells respond to glucose and other secretagogues. TCB f-MΦ was treated with 1 μg/ml LPS+25 mM glucose for 3 days and used for testing insulin release. Data represent one of at least three experiments with the similar results.

### DETAILED DESCRIPTION OF THE INVENTION

The present invention relates to pluripotent stem cells derivable from cord blood, as well as methods for culturing, propagating and/or differentiating such cells. The invention also provides methods of using such cells to treat any of a variety of disorders or disorders or diseases, or to ameliorate at least one symptom of one or more such disorders or diseases. The pluripotent adult stem cells of the invention are a subset of monocytes and are preferably obtained from humans. The cells of this subset are herein identified as monocyte-derived stem cells. Furthermore, as will be provided in the examples, the monocyte-derived stem cells are further derived from human cord blood. The examples provided herein demonstrate that a cord blood monocyte derived stem cell can be induced to differentiate into a variety of non-terminally or terminally differentiated cells, including endothelial cell, insulin-expressing cell, and macrophage (i.e., to acquire a phenotype characteristic of such a cell).

One advantage of the present invention is the capability to administer autologous cord blood monocyte derived stem cells, and/or cells differentiated therefrom, to patients in need of such cells. It will soon no longer be commonplace to simply discard umbilical cord blood and all of its valuable cells after the birth of a child. Indeed, umbilical cord blood is often stored or banked after birth. The use of autologous cord blood monocyte derived stem cells or their progeny reduces the risk of immune rejection and the transmission of disease. Additionally, umbilical cord blood has the advantage of tolerance for a degree of hman leukocyte antigen (HLA) incompatibility not possible with adult bone marrow, resulting in greater likelihood of finding an appropriate match. Further the ability to propagate autologous cord blood monocyte derived stem cells to useful quantities is expected to expand the number and variety of disorders and diseases amenable to therapies (and the number and variety of symptoms thereof amenable to amelioration) based on cord blood monocyte derived stem cells administration. The dosage and manner of administration are readily determinable by one of skill in the art using nothing more than routine optimization, with such efforts being guided by the type of cells being administered (cord blood monocyte derived stem cells, and/or derivatives thereof). Thus, the ability to store, propagate and differentiate the cord blood monocyte derived stem cells make them invaluable for autologous administration.

Advantages of the present invention include the use of umbilical cord blood as a convenient source for cord blood monocyte derived stem cells, including autologous cord blood monocyte derived stem cells, which can be safely and economically obtained by M-CSF treatment and TPO expansion. To better understand the invention, the following definitions are provided.

“Adult” or “adult human” means a mature organism or a mature cell such as a mature human or a mature human cell, regardless of age, as would be understood in the art.

The term “stem cell” refers to any cell that has the ability to differentiate into a variety of cell types, including terminally differentiated cell types. Such cells are, therefore, properly regarded as progenitor cells. Stem cells can be pluripotent, i.e., capable of differentiating into a plurality of cell types. An example of a stem cell is the herein described f-MΦ, or CB f-MΦ, or TCB f-MΦ (TPO-expanded CB f-MΦ).

As defined herein, the term “isolated” refers to cells that have been removed from their natural environment, typically the body of a mammal. Preferably, isolated cells are separated from other cell types such that the sample is homogeneous or substantially homogeneous. As a specific example, a blood cell monocyte is isolated if it is contained in a sample of blood that has been removed from an organism.

“Monocyte-derived stem cell” means stem cell derived from the monocyte fraction of the blood. “Cord blood monocyte” means a monocyte cell typically found in the umbilical cord blood of a vertebrate such as a mammal. These definitions comport with the ordinary and accustomed meanings of these cell-based terms in the art.

“Surface antigen” means a compound, typically proteinaceous, that is capable of binding to an antibody and is typically localized to a cell surface, such as by association with a cell membrane. A cell “marker,” such as a “macrophage marker,” is a detectable element sufficiently associated with a cell, such as a macrophage, as to be characteristic of that cell or cell type. One class of useful markers is cell-surface markers, which can be detected with minimal disruption of cellular activity.

Cell-based “activity” refers to a function(s) of a given cell or cell type. One category of useful activities is the activities useful in distinguishing a given cell or cell type from other cells or cell types. For example, an activity of a macrophage is phagocytosis, which is a distinguishing characteristic of macrophages.

“Cytokine” is given its ordinary and accustomed meaning of a regulatory protein released by a cell usually of the immune system that acts as an intercellular mediator in the generation of a cellular response such as an immune response. Examples of cytokines are the interleukins and lymphokines.

“Dispersion” means dissolution, i.e., to loosen or dissociate. As used herein, dispersion is not limited to dissolving or forming a solution thereof. In the context of the invention, the dissociation of cells, or a cell and a solid surface, typically a solid surface available to the cell during cell culture or propagation.

“Vertebrate” is given its ordinary and accustomed meaning of any organism properly characterized as having a bony or cartilaginous backbone made of vertebra. Similarly, the term “mammalian,” as defined herein, refers to any vertebrate animal, including monotremes, both marsupial and placental, that suckle their young and either give birth to living young (eutharian or placental mammals) or are egg-laying (metatharian or nonplacental mammals). Examples of mammalian species include primates (e.g., humans, monkeys, chimpanzees, baboons), rodents (e.g., rats, mice, guinea pigs, hamsters, rabbits), ruminants (e.g., cows, horses, sheep), canines (e.g., dogs, wolves) and felines (e.g., lions, tigers, cats).

By “suitable conditions” for growth, propagation or culture, it is meant that the temperature, humidity, oxygen tension, medium component concentrations, time of incubation and relative concentrations of cells and growth factors are at values compatible with the generation of progeny or sustaining cell viability. Each of the variables involved in cell growth or culture is well known in the art and, generally, a range of suitable values can be obtained using routine experimentation to optimize each result-effective variable.

The term “growth” is given its ordinary and accustomed meaning of the expansion of a cell population and/or cell size. Thus, the term “growth factor” as defined herein refers to a compound that is capable of inducing, or modifying the rate of, cell growth.

A cell “culture” is one or more cells within a defined boundary such that the cell(s) are allotted space and growth conditions typically compatible with cell growth or sustaining its viability. Likewise, the term “culture,” used as a verb, refers to the process of providing said space and growth conditions suitable for growth of a cell or sustaining its viability.

The term “propagate” or “propagation” refers to the process of cell growth. A “mitogenic compound” is a compound capable of affecting the rate of cell division for at least one cell type under at least one set of conditions suitable for growth or culture.

The phrase “disorder amenable to cell-based treatment” refers to a disorder that can be treated in whole or in part by administration of cells, whether autologous or heterologous to the recipient. The definition further embraces those disorders characterized by an effective cell deficiency (e.g., deficiency in number of cells or deficiency in number of healthy cells) as well as those disorders resulting from an abnormal extracellular signal wherein the administered cells can modulate/affect the level of that signal. As such, the definition embraces the physical re-supplying of cells and/or taking advantage of the physiology of the administered cells to restore an extracellular signal to levels characteristic of, or approaching that of, healthy individuals.

The term “differentiation” is given its ordinary and accustomed meaning of the process by which a cell or cells change to a different and phenotypically distinct cell type. A “differentiation inducer” is a compound that is a direct, or indirect, causative agent of the process of cell differentiation. Using this definition, a “differentiation inducer” is not be essential to differentiation.

An “inducing agent” or inducer is a differentiation inducer, i.e., a substance capable of directing, facilitating or promoting at least one type of cellular differentiation.

An “age-related CNS change” means a central nervous system alteration or change as manifested by confusion regarding the current time, the current date, the current location, self-identity, recent memory loss, or one or more other common facts that are well known and provide a basis for assessing the mental state of humans.

An “effective” or “pharmaceutically effective” amount is that amount that is associated with a desired effect, for example a pharmaceutical effect. Typically in the context of the invention, it is that amount or number of cord blood derived stem cells (and/or differentiated cord blood stem cell derivatives) which, when administered using conventional techniques, will result in a beneficial effect on a disorder or disease, or a symptom associated therewith, without unacceptably deleterious effects on the health or well being of the animal or human patient. By way of example, an effective amount is that amount of M-CSF that causes cord blood monocyte propagation, and particularly cord blood monocyte derived stem cell propagation, preferably increasing the relative contribution of cord blood monocyte derived stem cells to such cultures. A pharmaceutically effective amount, by way of example, is that amount of insulin-expressing cells derived from cord blood monocyte derived stem cells that will ameliorate a symptom of diabetes.

“Viability” is given its ordinary and accustomed meaning of a state characterized by the capacity for living, developing or germinating. In context, “viability” refers to the state of a cell. Measures of viability include, but are not limited to, a determination of the absolute, or relative, number(s) of cells, or an assessment of the absolute or relative health of one or more cells, using any one or more characteristic or property of a cell recognized in the art as informative on the health of a cell.

In view of the preceding definitions, one of ordinary skill in the art will appreciate that the invention provides methods for preparing an isolated cord blood monocyte derived stem cells that comprise the steps of (a) isolating a cord blood monocyte, (b) contacting the cord blood monocyte with an effective amount of a mitogenic compound selected from the group consisting of macrophage colony-stimulating factor (M-CSF), interleukin-6, human recombinant interleukin-12, β-nerve growth factor, vascular endothelial growth factor isoform 165, hepatocyte growth factor (HGF), phorbol 12-myristate 13-acetate (PMA), lipopolysaccharide (LPS), and leukemia inhibitory factor (LIF), and (c) culturing the cord blood monocyte under conditions suitable for propagation of the cell, thereby obtaining a preparation of an isolated cord blood monocyte derived stem cell.

An isolated cord blood monocyte is incubated with an effective amount of M-CSF (25-200 ng/ml), IL-6 (10-50 ng/ml) or LIF (100-2000 units/ml) according to one aspect of the invention. Preferably, 50 ng/ml M-CSF, 20 ng/ml IL-6 or 1000 units/ml LIF is used to treat preparations of cultured human umbilical cord blood monocytes.

The M-CSF, IL-6, IL-12 p70, LIF, or IL-1β used in the invention may be from any suitable source, such as a natural or synthetic source, and may be used in a purified or unpurified state. Further, it is contemplated that the M-CSF, IL-6, IL-12 p70, LIF, or IL-1β may be a holoprotein or may be active subunits or fragments that exhibit a mitogenic effect on cord blood monocytes. Similarly, the M-CSF, IL-6, IL-12 p70, LIF, or IL-1β may be used alone or in combination (e.g. with other mitogens) with suitable buffers and the like. The use of conventional assays may be used to determine the quantity and dosage of M-CSF, IL-6, IL-12 p70, LIF, or IL-1β associated with a sufficient mitogenic effect.

According to the methods of the invention, cord blood monocytes are incubated with one or more growth factors (i.e. mitogenic compounds) under suitable growth conditions to propagate cord blood monocyte derived stem cells. Likewise, the cord blood monocyte derived stem cells of the present invention may be incubated with one or more of various differentiation inducers (i.e. inducers or inducing agents), and optionally one or more growth factors, under suitable conditions to allow for differentiation, and optionally propagation, of a variety of cell types. As one of ordinary skill would recognize, there are known compounds that function as both growth factors and differentiation inducers. Growth factors of the invention include but are not limited to M-CSF, IL-6, LIF, and IL-12. Examples of compounds functioning as growth factors and/or differentiation inducers include, but are not limited to, lipopolysaccharide (LPS), phorbol 12-myristate 13-acetate (PMA), stem cell growth factor, human recombinant interleukin-2 (IL-2), IL-3, epidermal growth factor (EGF), b-nerve growth factor (bNGF), recombinant human vascular endothelial growth factor ₁₆₅ isoform (VEGF₁₆₅), and hepatocyte growth factor (HGF). Useful doses for inducing cord blood monocyte derived stem cells differentiation by growth and/or differentiation factors are: 0.5 ng/ml-1.0 μg/ml (preferably 1.0 μg/ml) for LPS, 1-160 nM (preferably 3 nM) for PMA, 500-2400 units/ml (preferably 1200 ng/ml) for bNGF, 12.5-100 ng/ml (preferably 50 ng/ml for VEGF), 10-200 ng/ml (preferably 100 ng/ml) for EGF, and 25-200 ng/ml (preferably 50 ng/ml) for HGF.

Cell surface antigens and cell markers may be identified using any technique known in the art, including immunostaining. Surface antigens and markers which, alone or in combination, are characteristic of cells according to the invention include MAC-1, CD117, CD14, CD34, CD40, C45, and CD163 whereas CD1a and CD83 are characteristically not associated with cells according to the invention. By way of example, cell surface antigens or markers have been identified using cells on glass slides, the cells having been immunostained by washing with phosphate-buffered saline (PBS) and fixed with 4% formaldehyde in PBS for 20 minutes at 20° C. For intracellular proteins, the cells were permeabilized with 0.5% Triton X-100 for 5 minutes at 20° C. and incubated for one hour with the primary antibodies. The primary antibodies were diluted with PBS containing 1% BSA to block non-specific reactivity. The cells were then washed 3 times with PBS containing 1% BSA and incubated for 45 minutes with FITC-, TRITC-, or CyS-conjugated cross-adsorbed donkey secondary antibodies (Jackson ImmunoResearch, West Grove, Pa.). Both of these reactions were performed at saturating concentrations and at 4° C. The slides were then washed and mounted with phosphate-buffered gelvatol.

Fluorescence imaging may be used to monitor or detect cells and is performed using techniques known in the art. For example, automated excitation and emission filter wheels, a quad-pass cube, and SlideBook software may be used for fluorescence imaging. Quantitative fluorescence ratio imaging can be performed using glyceraldehyde 3-phosphate dehydrogenase immunofluorescence (sheep polyclonal antibody, Cortex Biochem., San Leonardo, Calif.) as an internal standard. The fluorescence intensity level detected after reacting a sample with an isotype-matched IgG antibody provides a background fluorescence level, which is primarily attributable to non-specific binding. This fluorescence intensity was arbitrarily assigned an intensity level of one.

Among the antibodies contemplated for use in the invention are mouse monoclonal antibodies to IL-1β, IL-6, IL-10, CD14, CD31, CD41a, CD41b, CD42b, CD163, CD34, CD40, CD45, HLA-DR, HLA-DQ, CD1a, CD83, von Willebrand's factor (vWF), keratins (Pan Ab-1), cytokeratin 7, α-fetoprotein (AFP), microtubule-associated protein-1B (MAP-1B), neurofilament Ab-1 (NF), IL-12p70, tumor necrosis factor-α (TNF-α), TNF-αreceptor I (TNF-RI) and TNF-RII. Further, mouse IgG₁, IgG_{2A}, IgG′_{2B}, and goat IgG antibody to CD3, CD4, CD8 and human albumin; rat monoclonal antibody to E-cadherin; rabbit polyclonal antibodies to neuron-specific enolase (NSE), peroxisome proliferator-activated receptor (PPAR)γ2, IL-6, leptin and VEGF-R3 (FLT-4), and mouse monoclonal antibody to VEGF-R2 (FLK-1) are also contemplated for use in the invention.

Upon incubation with the appropriate differentiation inducer, a cord blood monocyte derived stem cell of the invention has the ability to differentiate into a variety of cell types. Furthermore, proliferation of a cord blood monocyte derived stem cell of the present invention can be effected by treatment with low dose thrombopoietin (preferably between 2.5 and 5 ng/ml). As will be illustrated in the examples, thrombopoietin possesses dual effects on the proliferation of cord blood monocyte derived stem cells. At lower dosage (2.5-5 ng/ml), thrombopoietin significantly increased cell number to a level that was about 2 times more than the untreated cord blood monocyte derived stem cells, and 10 times higher than the control monocytes.

For example, according to methods of the invention, following contact by an effective amount of lipopolysaccharide in the presence of 25 mM glucose, a cord blood monocyte derived stem cell differentiates into an insulin-expressing cell. In one embodiment, 1 μg/ml of LPS in the presence of 25 mM glucose was used to treat cord blood monocyte derived stem cells having been treated with thrombopoietin at low doses (preferably between 2.5 and 5 ng/ml). In another option, 1 μg/ml of LPS in the presence of 25 mM glucose may be used to treat cord blood monocyte derived stem cells not having been treated with thrombopoietin at low doses.

According to other methods of the invention, following contact by an effective amount of bNGF, a cord blood monocyte derived stem cell differentiates into a neuronal cell when under suitable growth conditions. In one embodiment, 200 ng/ml bNGF was used to treat cord blood monocyte derived stem cell cultures. It is contemplated by the invention that inducers of neuronal cell differentiation known in the art may be used under growth conditions and inducer concentrations that allow for optimal differentiation. These may include, but are not limited to, NGF, brain-derived neurotrophic factor, neurotrophin-3, basic fibroblast growth factor, pigment epithelium-derived factor, or retinoic acid.

According to still other methods of the invention, endothelial cells are prepared by contacting cord blood monocyte derived stem cells with VEGF under suitable growth conditions. In one embodiment, 50 ng/ml of VEGF was used to treat cultures of cord blood monocyte derived stem cells for 5-7 days. However, it is contemplated by the invention that other known inducers of endothelial cell differentiation may be substituted for VEGF. These may include, but are not limited to, insulin growth factor and basic fibroblast growth factor. In another embodiment, 50 ng/ml of VEGF was used to treat cultures of cord blood monocyte derived stem cells for 5-7 days after the cells were treated with thrombopoietin in low dose (preferably between 2.5 and 5 ng/ml).

Analogously, the invention provides methods to prepare epithelial cells by contacting cord blood monocyte derived stem cells with EGF under suitable culture conditions. By way of example, 100 ng/ml EGF was incubated with a cord blood monocyte derived stem cell sample for 4 days. However, it is contemplated by the invention that other known inducers of epithelial cell differentiation may be substituted for EGF. These include, but are not limited to, bone morphogenesis protein-4, elevated calcium concentrations, retinoic acid, sodium butyrate, vitamin C, hexamethylene bis acetate, phorbol 12-myristate 13-acetate (PMA), teleocidin, interferon gamma, staurosporin, or activin.

According to still other methods of the invention, a macrophage and/or a T-lymphocyte is prepared by contacting a cord blood monocyte derived stem cell with an appropriate inducer, such as LPS, for macrophage development and IL-2 for T-lymphocyte development. For example, 1 μg/ml LPS and 1200 units/ml IL-2 are incubated with cord blood monocyte derived stem cells to achieve macrophage and T-lymphocyte cell differentiation, respectively. It is contemplated by the invention that other known inducers of macrophage and T-lymphocyte cell differentiation may be substituted for LPS and IL-2. These may include, but are not limited to, IL-4, IL-12, IL-18, CD3 antibody, PMA, teleocidin, or interferon gamma.

In a similar way, the invention provides methods to prepare hepatocytes by contacting cord blood monocyte derived stem cells with human recombinant hepatocyte growth factor (HGF) under suitable culture conditions. By way of example, 50 ng/ml of HGF is incubated with a cord blood monocyte derived stem cell sample for 5-7 days. However, it is contemplated by the invention that other known inducers of hepatocyte differentiation may be substituted for HGF. These include, but are not limited to, retinoic acid, oncostatin M, phenobarbital, dimethyl sulfoxide, dexamethasone, or dexamethasone and dibutyryl cyclic AMP.

The currently described cord blood monocyte derived stem cell and/or cell derived therefrom is, among other uses, employed to replenish a cell population that has been reduced or eradicated by a disease or disorder (e.g., cancer), by a treatment for such a disease or disorder (e.g., a cancer therapy), or to replace damaged or missing cells or tissue(s). By way of example, neuronal tissue damaged during the progression of Parkinson's disease, endothelial cells damaged by surgical incisions, macrophage cells affected by Gaucher's disease, epithelial cells damaged from skin burns, T-lymphocytes affected by Lyme disease or hepatocytes damaged as a result of cirrhosis, are replenished by cells according to the invention. In addition, individuals with congenital diseases can be engrafted with autologous cord blood monocyte derived stem cells or their progeny, after repairing the genetic alteration or further modifying the genome (e.g., introduction, deletion or modification of an expression control sequence, introduction of a modification in the genome that functions as a second-site reversion, and the like) by recombinant technology. Moreover, the ability to propagate autologous cord blood monocyte derived stem cells in vitro before administration of such cells should yield a sufficient number of stem cells for this procedure, which is expected to be more effective and versatile than the current transplantation procedures that do not include such an expansion.

Having now generally described the invention, the same will be more readily understood through reference to the following Examples which are provided by way of illustration, and are not intended to be limiting of the present invention.

### EXAMPLE 1

### Generation of f-MΦ from Human Umbilical Cord Blood

Human umbilical cord blood-derived monocytes were treated with 50 ng/ml M-CSF for 10-14 days. After this treatment, approximately 80-90% of the cells were elongated. To confirm they were f-MΦ, cell markers were evaluated. Immunostaining demonstrated that these cells expressed specific macrophage marker CD14, leukocyte common antigen CD45, and hematopoietic stem cell markers CD34 and CD117. Cord blood derived monocytes were treated with 50 ng/ml M-CSF for 14 days and then used for staining. Cells express the f-MΦ surface markers including CD14, CD45, CD34, and CD117.

Double staining showed that these cells co-expressed macrophage functional markers, such as scavenger receptor CD163 and phagocytosis, demonstrated by phagocytosis of Dextran 10,000. Merged imaging showed the colocalization of double staining (yellow), wherein cells display the functional markers phagocytosis (red) and scavenger receptor CD163 (green). Phagocytosis was shown by phagocytizing Alexa Fluor 647-labeled Dextran 10,000.

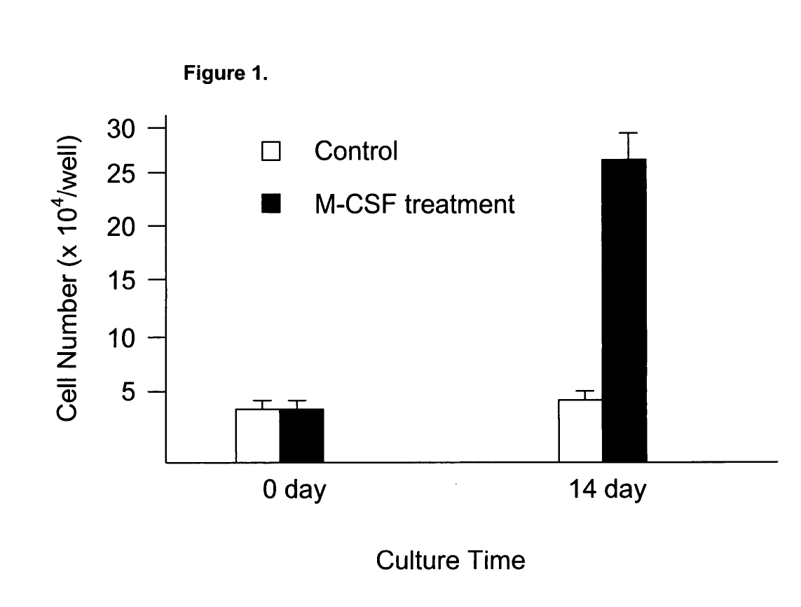

M-CSF treatment also significantly increased the cell number. See FIG. 1. These cellular characteristics are identical to those characterized f-MΦ isolated from human peripheral blood. See Zhao et al., Proc. Natl. Acad. Sci. USA 100(2003) 2426-2431. Most of the cells in untreated cultures demonstrated round attached morphology, displaying typical macrophage markers (CD14, CD45, CD163, and phagocytizing Dextran 10,000), but failing to express CD34 and CD117. The above experiments were performed in 4 separated cord blood samples with the similar results. The results indicate that the previously characterized f-MΦ can be generated from precursor in human umbilical cord blood.

### EXAMPLE 2

### Thrombopoietin (TPO) Stimulates the Proliferation of Human Cord Blood-Derived f-Macrophages (CB f-MΦ)

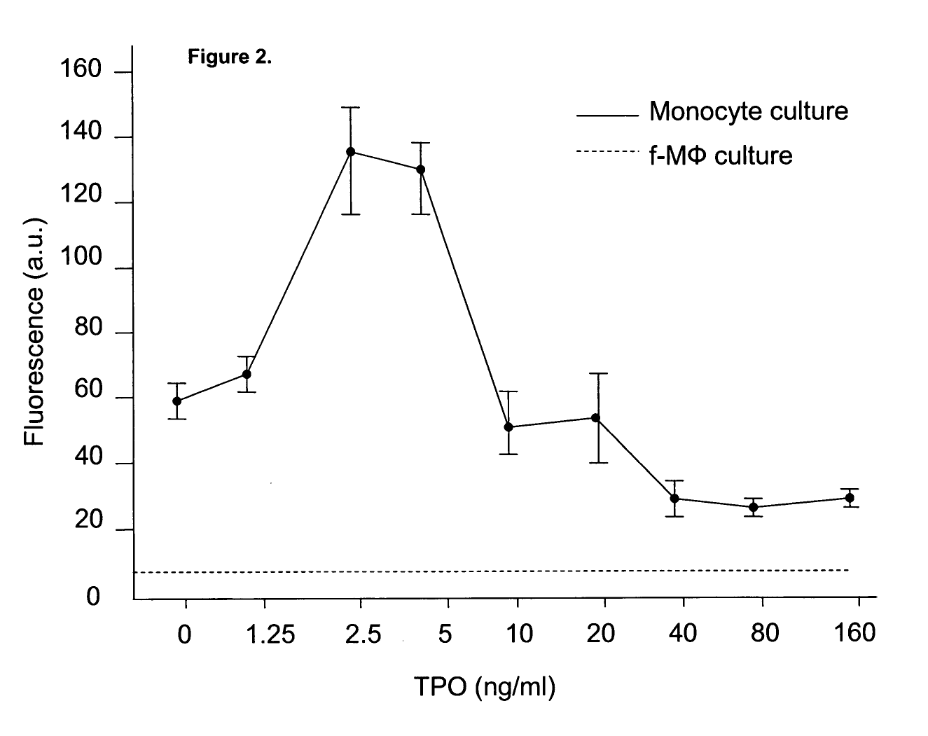

TPO modulates multiple aspects of hematopoiesis, including stem cell survival, self-renewal and expansion. Different dosages of TPO were evaluated for effects on CB f-MΦ. The results showed that TPO possessed dual effects on the proliferation of the CB f-MΦ. See FIG. 2. At lower dosage (2.5-5 ng/ml), TPO significantly increased cell number (FIG. 2), to a level that was about 2 times more than the TPO-untreated CB f-MΦ, and 10 times higher than the control monocytes. However, at higher dosage (>40 ng/ml), TPO inhibited the proliferation of CB f-MΦ. Low dose (5 ng/ml) TPO was further evaluated to increase the CB f-MΦ number in additional experiments.

### EXAMPLE 3

### TPO-Expanded CB f-MΦ (TCB f-MΦ) Retain Phenotypic Characteristics of f-MΦ

Retention of the original phenotype is a major concern after expanding stem cell cultures. Using 5 ng/ml TPO, CB f-MΦ could be passaged 5-8 times in RPMI 1640 medium supplemented with 3.5% FBS. After continuing culture for 2 months, immunostaining showed that TPO-stimulated cells continued to express leukocyte common antigen CD45, macrophage markers CD 14 and CD 163, and stem cell marker CD117 at the same level as parental CB f-MΦ. While CD34 expression was decreased to ˜30% of cells. FITC-conjugated secondary antibodies were used for CD45, CD117, and CD163, while FITC-conjugated antibody was used for CD14. Cells were photographed using Zeiss LSM 510 META confocal microscope.

TPO is also a key inducer for differentiation of platelets and megakaryocytes. To evaluate if such differentiation occurred after expansion of CB f-MΦ with TPO, the 4th and 5th passaged cells were examined with the specific platelet and megakaryocyte markers such as CD41a, CD41b, and CD42b. Results showed that expression of these markers was not induced after treatment with TPO.

### EXAMPLE 4

### Endothelial-Like Cell Differentiation of the TCB f-MΦ

Another major concern with stem cell expansion is the retention of multiple differentiation ability. To examine whether the CB f-MΦ retained their pluripotence after TPO expansion, their potential to display 2 diverse phenotypes using 4th and/or 5th passaged cells was evaluated. To examine whether the TCB f-MΦ retains the ability to differentiate into endothelial-like cells, cells were treated with 50 ng/ml VEGF for 5-7 days. Expression of the endothelial cell-associated markers Flt-1 (VEGF receptor 1), Flk-1 (VEGF receptor 2), von Willebrand Factor (vWF), and CD31 was then examined. The results showed that VEGF-treated cells significantly expressed Flt-1, Flk-1, vWF, and CD31. Functional analysis showed that both the VEGF-treated and VEGF-untreated TCB f-MΦ possessed strong ability to ingest the acetylated low density lipoprotein (Dil-Ac-LDL). However, the VEGF-untreated TCB f-MΦ was negative with Flt-1, vWF, and CD31 staining, with weak staining for Flk-1. The VEGF-treated or untreated f-MΦ was stained with endothelial cell markers Flt-1, Flk-1, von Willebrand Factor (vWF), CD31, and incorporated the acetylated low density lipoprotein (Ac-LDL).

After VEGF treatment for 5-7 days, cellular morphology changed from the spindle-like f-MΦs to broad endothelial-like cells with spontaneous formation of chain-like structures. These structures were further characterized with specific endothelial cell marker CD31 and endocytosis of Ac-LDL and confirmed that they were double positive. The cells were double stained with Ac-LDL (red) and CD31 (green) and then merged with the differential interference contrast (DIC) image (yellow). The isotype-matched IgG1 mAB served as the negative control.

Taken together, the above observations demonstrate that the TCB f-MΦ can maintain their ability to differentiate into endothelial-like cells after TPO expansion.

### EXAMPLE 5

### TCB f-MΦ Differentiate into Insulin-Expressing Cells

According to current developmental theory, blood cells and endothelial cells are derived from the same mesoderm layer. Therefore, to further test whether TCB f-MΦ possess pluripotent differentiation potential, their capacity to produce insulin-expressing cells, which develop from the endoderm layer, was evaluated. To date, nestin as an intermediate filament protein has been regarded as the marker of a neuroendocrine progenitor and nestin-positive cells can give rise to insulin-producing cells. CB f-MΦ was initially stained with a human nestin monoclonal antibody. The results showed that all the CB f-MΦ strongly expressed nestin both before and after TPO expansion, suggesting that CB f-MΦ may possess the potential to differentiate to insulin-positive cells.

TCB f-MΦ was treated with the macrophage activator lipopolysaccharide (LPS, 1 μg/ml) in the presence of 25 mM glucose. Subsequent to this treatment, the morphology of cells changed from an elongated to a round attached morphology. Immunostaining with insulin and C-peptide (a by-product of insulin) antibodies demonstrated that 85-90% of cells were strongly positive.

Analyzing with confocal microscopy revealed that there were insulin-positive granules in the cytoplasm of insulin-positive cells. However, the untreated TCB f-MΦ was negative or showed background level with insulin and C-peptide staining. Nuclear staining with DAPI in these insulin-positive cells was next performed. The nuclear size and morphology showed no significant difference between the insulin-positive cells and untreated cells, suggesting that there was no apoptosis (insulin monoclonal antibody (green) and DAPI (blue) provided for double stained images of LPS-treated and untreated f-MΦ; not shown).

To further confirm that the insulin production was not due to uptake from serum in the culture medium, in situ hybridization with a human insulin oligonucleotide probe was performed. The results proved that insulin mRNA was strongly expressed in these insulin-immunostaining positive cells. The untreated TCB f-MΦ only showed very weak or negative hybridization (insulin mRNA expression was demonstrated by in situ hybridization; cells were derived from 1 μg/ml LPS-treated TCB f-MΦ for 3 days in the presence of 25 mM glucose and the untreated TCB f-MΦ.

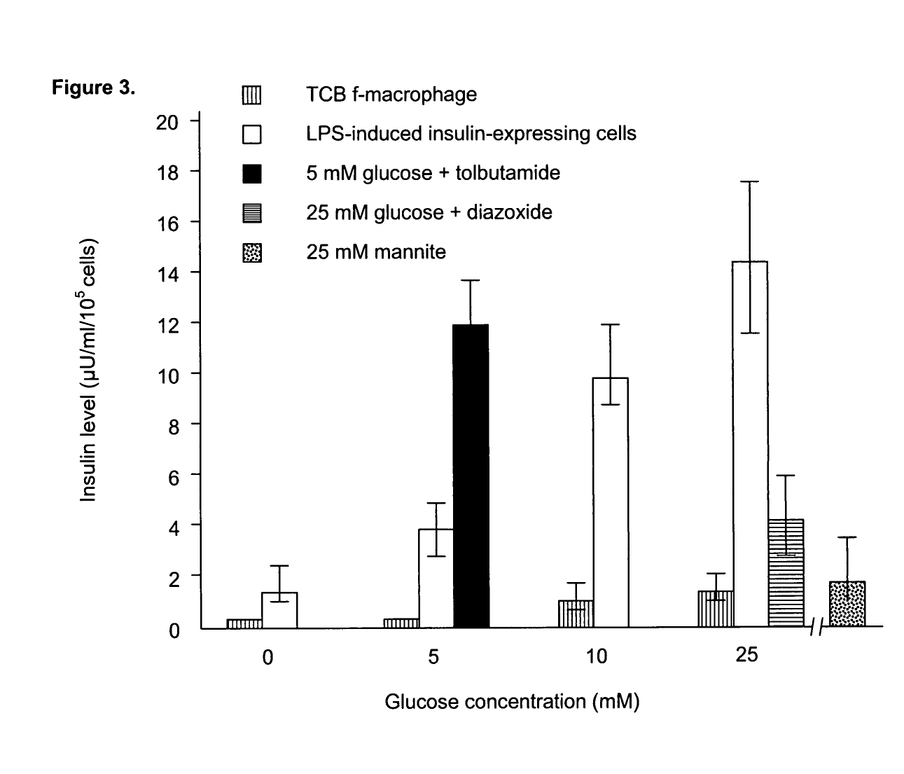

In vitro functional analysis demonstrated that these insulin-immunostaining positive cells could release insulin in a glucose-dependent manner (FIG. 3) and also respond to other secretagogues and inhibitor, such as tolbutamide (a sulfonylurea inhibitor of K_{ATP} channel) which stimulates insulin release in the presence of 5 mM glucose, or diazoxide (a classical K_{ATP} channel opener) which inhibits insulin release in the presence of 25 mM glucose (FIG. 3). To exclude the possibility of high osmolarity affecting insulin release, 25 mM mannite was used to generate similar osmolarity as 25 mM glucose, but it failed to stimulate insulin release in these cells (FIG. 3). The results indicate that TCB f-MΦ differentiated into the insulin-expressing cells after treatment with LPS combined with high glucose.

CB f-MΦ were also found to display glucagon-like peptide 1 (GLP-1) receptor after treatment with lipopolysaccharide (LPS). Immunostaining demonstrated that around 70% of treated f-MΦ expressed glucagon-like peptide 1 receptor. To optimize their differentiation into insulin-producing cells and improve their therapeutic potential, exendin-4 may be administered in combination with high glucose and/or lipopolysaccharide (LPS). Cells were treated with 50 ng/ml LPS in the presence of 25 mM glucose for 2 days in 8-well LabTeck II chamber slides and used for immunostaining with rabbit anti-human GLP-1 receptor polyclonal antibody. Normal rabbit IgG served as control for immunostaining.

### EXAMPLE 6

### Cord Blood-Derived Insulin Producing Cells Reverse Hyperglycemia in Diabetic Mice

To investigate the capacity of cord blood-derived insulin-producing cells (BDIPC) to correct hyperglycemia, BDIPC (2×10⁶ cells/mouse) were transplanted into renal capsule of the streptozotocin (“STZ”)-induced diabetic Balb/c nude mice. Five STZ-induced diabetic mice received physiological saline after sham surgery without cellular implantation as a control. Blood glucose levele in control mice remained very high (>500 mg/dl). BDIPC-transplanted mice showed decreasing of blood glucose level and retained a low level for 3 days (ranging from 230 to 280 mg/dl). These results indicate that the BDIPC were functional in vivo and capable of reversing hyperglycemia in diabetic mice.

### EXAMPLE 7

### Human Umbilical Cord Blood-Derived Fibroblast-Like Macrophage (f-MΦ) Display Photoreceptors

Immunostainings showed that >95% of f-MΦ expressed photoreceptor recoverin, and around 80% of f-MΦ expressed rhodopsin.

### EXAMPLE 8

### Materials and Methods Used in Conjunction with Foregoing Examples 1 through 7

Reagents

Recombinant human macrophage colony-stimulating factor (M-CSF), thrombopoietin (TPO), lipopolyssacharide (LPS), and dextrose (glucose) were purchased from Sigma (St. Louis, Mo.), respectively. Recombinant human vascular endothelial cell growth factor (VEGF) was obtained from Cell Sciences, Inc. (Canton, Mass.). Mouse anti-human monoclonal antibodies to CD31, CD41a, CD41b, CD42b, CD45, CD163, and IgG₁K immunoglobulin isotype control, FITC-conjugated CD 14 were all obtained from BD PharMingen (San Diego, Calif.). Mouse anti-human CD34 monoclonal antibody was from Vector Laboratories (Bur-lingame, Calif.). The mouse anti-human nestin and FITC labeled goat anti-rabbit IgG (H+ L) were obtained from Neuromics Antibodies (Northfield, Minn.). Mouse anti-human insulin monoclonal antibody and rabbit anti-human von Willebrand factor antiserum were obtained from Sigma. Rabbit anti-human C-peptide antiserum was from Linco Research (St. Charles, Mo.). Rabbit anti-human CD117/c-Kit, Flt-11VEGFR1, and Flk-1/KDR/VEGFR2 polyclonal antibodies were purchase from Lab vision corporation (Dr. Fremont Calif.), respectively. FITC labeled AffiniPure Donkey anti-mouse IgG antibody was obtained from Jackson ImmunoResearch Laboratories, Inc. (West Grove, Pa.). Polyclonal antibodies to photoreceptor recoverin and rhodopsin were purchased from Chemicon Internal (Temecula, Calif.).

Cell Culture

Human umbilical cord blood samples (50-100 ml/unit) were obtained from healthy donors (Life-Source Blood Services, Glenview, Ill.). The f-MΦ culture was performed as previously described using human peripheral blood (14). Briefly, purified monocytes were seeded into the 8-Well Lab-Tek II Chamber Slide (Fisher Scientific) at 1×10⁵ cells/ml, 0.5 ml/well in RPMI 1640 medium supplemented with 7% fetal bovine serum (Invitrogen, Carlsbad, Calif.) and 50 ng/ml M-CSF. The purity of f-MΦ normally reached 80-90% after incubation for 10-14 days at 37° C., 8% CO₂ and was then used for experiments.

For f-MΦ expansion, the cells at 70% confluence were passaged at ratio 1:2 by pippetting and gently scraping with 100 μl tips (Rainin, Woburn, Mass.). Media were changed every 5-7 days with the fresh RPMI 1640 medium supplemented with 3.5% fetal bovine serum and 5 ng/ml thrombopoietin (TPO).

Phagocytosis

Phagocytosis of f-MΦ was performed as previously described with minor modifications [J. A. Swanson, M. Lee, P. E. Knapp, Cellular dimensions affecting the nucleocytoplastnic volume ratio, J. Cell Biol. 115 (1991) 941-948]. Cells were incubated with 3.5 mg/ml of 10 kDa Dextran Alexa Fluor 647 (Molecular Probes Inc., Eugene, Oreg.) at 37° C., 8% CO₂ conditions for 3 h in the 8-well Lab-Tek chamber slide. After 5-8 times washing with PBS, the cells were fixed in 4% formaldehyde in PBS and mounted with Mounting Medium (Vector Laboratories, Burlingame, Calif.). The cells were viewed and photographed using Zeiss LSM 510 META confocal microscope equipped with a 25× water immersion objective (Carl Zeiss Inc.), with HeNe 633 nm laser beam filtered through LP650. The images were acquired with the manufacturer's software and edited using Adobe Photoshop Elements 2.0.

Cell Proliferation

Previous studies showed that f-MΦ became independent of M-CSF stimulation after 10 days [Y. Zhao, D. Glesne, E. Huberman, A human peripheral blood monocyte-derived subset acts as pluripotent stem cells, Proc. Natl. Acad. Sci. U.S.A. 100 (2003) 2426-2431]. Therefore, this stage f-MΦ, at ˜80% purity, was selected to examine the effect of TPO. Cells were washed with RPMI 1640 medium three times and then incubated with different concentration of TPO, such as 80, 40, 20, 10, 5, 2.5, 1.25, 0.625, and 0 ng/ml in the 8-well Lab-Tek chamber slides (Nunc, Naperville, Ill.) at 37° C., 8% CO₂ conditions. After 4 days, cell proliferation was evaluated using CyQUANT® Cell Proliferation Assay Kit (Molecular Probes, Eugene, Oreg.). Cell fluorescence was measured using a Synergy HT Multi-Detection microplate reader (Bio-Tek Instruments Inc., Winooski, Vt.) equipped with filters for ˜480 nm excitation and ˜520 nm emission. The optical values were analyzed using the manufacturer's software KC4 v3.1.

Immunostaining

Immunostaining was performed as previously described with minor modifications [Y. Zhao, D. Glesne, E. Huberman, A human peripheral blood monocyte-derived subset acts as pluripotent stem cells, Proc. Natl. Acad. Sci. U.S.A. 100 (2003) 2426-2431]. The cells were incubated for 20 min at room temperature with 2.5% diluted normal horse serum (Vector Laboratories, Burlingame, Calif.) to block nonspecific staining. Cells were then incubated with primary antibodies, the isotype-matched IgG_{1κ} (BD PharMingen, San Diego, Calif.) (as control for mouse monoclonal antibodies), and normal rabbit serum (as control for rabbit polyclonal antibodies). After staining, the slides were mounted with Mounting Medium (Vector Laboratories, Burlingame, Calif.). Cells were viewed and photographed using a Zeiss LSM 510 META confocal microscope equipped with a 25× water immersion objective (Carl Zeiss Inc.), with Argon 488 nm laser for FITC fluorochrome filtered through LP505. The images were acquired with the manufacturer's software and edited using Adobe Photoshop Elements 2.0.

For double staining assays, cells first completed phagocytizing Dextran 10,000 or incorporating Ac-LDL, and then were fixed with 4% formaldehyde for 20 min at room temperature and used for immunostaining with specific cell surface markers as described above. For insulin together with 4′,6-Diamidino-2-phenylindole (DAPI) nuclear staining, cells first completed single staining with insulin monoclonal antibody as described above, and then mounted the slide with VECTSHIELD Hard Set Mounting Medium with DAPI (Vector Laboratories). Two fluorochromes were scanned sequentially by using a multi-tracking function to reduce any bleed-through artifacts.

Cell Differentiation and Characterization

The TPO-expanded CB f-MΦ was treated with 50 ng/ml VEGF (Cell Sciences Inc., Canton, Mass.) in RPMI-1640 medium supplemented with 7% heat inactivated FBS as previously described [Y. Zhao, D. Glesne, E. Huberman, A human peripheral blood monocyte-derived subset acts as pluripotent stem cells, Proc. Natl. Acad. Sci. U.S.A. 100 (2003) 2426-2431]. After 5-7 days, the treated and untreated cells were prepared for characterizing with endothelial markers, including antibodies to Flt-1, Flk-1, vWF, CD31, and incorporating Dil-Ac-LDL as previously described [D. A. Ingram, L. E. Mead, H. Tanaka, V. Meade, A. Fenoglio, K. Mortell, K. Pollok, M. J. Ferkowicz, D. Gilley, M. C. Yoder, Identification of a novel hierarchy of endothelial progenitor cells using human peripheral and umbilical cord blood, Blood 104 (2004) 2752-2760].

The TPO-expanded CB f-MΦ was treated with 1 μg/ml LPS in RPMI-1640 medium supplemented with 7% heat inactivated FBS and 25 mM glucose. The final insulin concentration in the culture medium was 0.196 μU/ml. After 3-5 days, the treated and untreated cells were prepared for characterizing insulin production at both protein and mRNA level by immunostaining and in situ hybridization, respectively.

In Situ Hybridization

The LPS-treated and untreated cells were fixed with 4% cold formaldehyde at 4° C. for 15 min. After blocking the endogenous peroxidase with ImmunoPure Peroxidase Suppressor (Pierce, Rockford, Ill.), the cells were used for in situ hybridization following the protocols provided by GeneDe-tect.com Ltd. (Bradenton, Fla.). The biotin-labeled oligonucleotide probes to human insulin antisense, sense, and poly d(T) were obtained from GeneDetect.com Ltd., respectively. The treated and untreated cells were incubated with the probes in hybridization buffer (DakoCytomation, Carpin-teria, Calif.) at dilution 1:100 (1 μg/ml), 37° C. for 16-18 h. The signals were detected using the Tyramide Signal Amplification System for In Situ Hybridizations by following their protocols as provided in the kit (DakoCytomation, Carpinteria, Calif.). The cells were viewed using a 40× oil immersion objective under the Zeiss Axioskop Histology/Digital Fluorescence microscope (Hallbergmoos, Germany) equipped with 0.63× coupling adapter, and then photographed with a Zeiss Axiocam Color Camera (Hallbergmoos, Germany) with 13,000×1030 pixels. The images were analyzed with the Zeiss AxioCam Plug-In for Photoshop software version 1.1 (Hallbergmoos, Germany).

Insulin Release

TPO-expanded CB f-MΦ (TCB f-MΦ) cultured in 24-well tissue culture plates (Costar, Corning, N.Y.) at 60-70% confluence was treated for 3 days with 1 μg/ml LPS+25 mM glucose in 1 ml 7% FBS-RPMI 1640 medium per well. TCB f-MΦ cultured in the same concentration of glucose served as control. Insulin release was performed as previously described with minor modifications [F. Schuit, A. De Vos, S. Farfari, K. Moens, D. Pipeleers, T. Brun, M. Prentki, Metabolic fate of glucose in purified islet cells. Glucose-regulated anaplerosis in beta cells, J. Biol. Chem. 272 (1997) 18572-18579; and N. Lumelsky, O. Blondel, P. Laeng, I. Velasco, R. Ravin, R. McKay, Differentiation of embryonic stem cells to insulin-secreting structures similar to pancreatic islets, Science 292 (2001) 1389-1394]. Cells were washed 5 times with PBS to remove the serum and then incubated with Kreb's buffer (120 mM NaCl, 5 mM KCl, 2.5 mM CaCl₂, 1.1 mM MgCl₂, 25 mM NaHCO₃, 0.1% BSA) [N. Lumelsky, O. Blondel, P. Laeng, I. Velasco, R. Ravin, R. McKay, Differentiation of embryonic stem cells to insulin-secreting structures similar to pancreatic islets, Science 292 (2001) 1389-1394] for 30 min in the absence of glucose, at 37° C., 8% CO₂ conditions. After removing the Kreb's buffer, cells were incubated with different concentration of glucose (0, 5, 10, and 25 mM) and/or other secretagogues (10 μM tolbudamide (Sigma), 500 μM diazoxide (Sigma), or 25 mM mannite (Sigma) in 300 μl Kreb's buffer/well for 1 h. Each treatment had duplicated wells. Insulin levels were monitored using an ultrasensitive human insulin enzyme-linked immunosorbent assay (ELISA) kit (Alpco Diagnostics, Windham, N.H.) following the manufacturer's protocols. Cell number/well from each treatment was finally quantified for calculating insulin level.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

We claim:

1. An isolated cord-blood monocyte derived stem cell, wherein the cell exhibits a surface antigen selected from the group consisting of MAC-1, CD14, CD34, CD40, CD45, CD117, and CD163.

2. The isolated cord blood monocyte derived stem cell of claim 1, wherein the cell exhibits phagocytic activity.

3. The isolated cord blood monocyte derived stem cell of claim 2, wherein the cell expresses CD163.

4. A method of preparing an isolated monocyte derived stem cell comprising the steps of:
(a) isolating a cord blood derived monocyte;
(b) contacting the cord blood derived monocyte with an effective amount of a mitogenic compound selected from the group consisting of macrophage colony-stimulating factor, interleukin-6, and leukemia inhibitory factor; and
(c) culturing the cord blood derived monocyte under conditions suitable for propagation of the cell, thereby obtaining a preparation of an isolated cord blood monocyte derived stem cell.

5. The method of claim 4, wherein the cord blood derived monocyte is cryopreserved prior to contacting the monocyte with a mitogenic compound.

6. The method of claim 4 further comprising cryopreserving the isolated cord blood monocyte derived stem cell.

7. The method of claim 4, wherein the cord blood derived monocyte is a mammalian cord blood derived monocyte.

8. The method of claim 4, further comprising
(d) culturing the isolated cord blood monocyte derived stem cell in thrombopoietin, thereby proliferating the isolated stem cell.

9. The method of claim 7, wherein the mammalian cord blood derived monocyte is a human cord blood derived monocyte.

10. An isolated cord blood monocyte derived stem cell obtained by the method of claim 4.

11. A method of generating a differentiated cell comprising the steps of:
(a) isolating a cord blood monocyte derived stem cell according to the method of claim 4; and
(b) contacting the cord blood monocyte derived stem cell with an amount of an including gent effective to induce differentiation of the cell, thereby generating a differentiated cell.

12. The method of claim 11, further comprising cryopreserving the differentiated cell.

13. The method of claim 11, further comprising culturing the differentiated cell.

14. The method of claim 13, wherein the differentiated cell/inducing agent are selected from the group consisting of a insulin-expressing cell/lipopolysaccharide plus glucose, neuronal cell/nerve growth factor (bNGF), an endothelial cell/vascular endothelial growth factor (VEGF), an epithelial cell/epidermal growth factor (EGF), a T-lymphocyte/interleukin-2 (IL-2), a macrophage/lipopolysaccharide (LPS), a hepatocyte/hepatocyte growth factor (HGF), and a differentiating medium for a retinal pigment epithelial cell (RPE).

15. A method for identifying a cell type-specific therapeutic agent comprising:
(a) contacting a first differentiated cell obtained according to the method of claim 11 and a candidate therapeutic agent;
(b) further contacting a second differentiated cell obtained according to the method of claim 10 and the candidate therapeutic agent, wherein the first and second differentiated cells are different cell types; and
(c) measuring the viability of the first differentiated cell relative to the viability of the second differentiated cell, wherein a difference in viabilities identifies the candidate therapeutic agent as a cell type-specific therapeutic agent.

16. A method of treating a disorder amenable to cell-based treatment comprising administering a pharmaceutically effective amount of a cord blood monocyte derived stem cell.

17. A method of treating an endothelial cell disorder amenable to cell-based treatment comprising administering a pharmaceutically effective amount of an endothelial cell obtained by the method of claim 14.

18. A method of treating an insulin-expressing cell disorder amenable to cell-based treatment comprising administering a pharmaceutically effective amount of an insulin-expressing cell obtained by the method of claim 14.

19. A method of treating neuronal cell disorder amenable to cell-based treatment comprising administering a pharmaceutically effective amount of a neuronal cell obtained by the method of claim 14.

20. A method of treating an epithelial cell disorder amenable to cell-based treatment comprising administering a pharmaceutically effective amount of an epithelial cell obtained by the method of claim 14.

21. A method of treating a T-lymphocyte disorder amenable to cell-based treatment comprising administering a pharmaceutically effective amount of T-lymphocyte obtained by the method of claim 14.

22. A method of treating a macrophage cell disorder amenable to cell-based treatment comprising administering a pharmaceutically effective amount of macrophage cell obtained by the method of claim 14.

23. A method of treating a hepatocyte cell disorder amenable to cell-based treatment comprising administering a pharmaceutically effective amount of a hepatocyte cell obtained by the method of claim 14.

24. A method of treating a photoreceptor cell disorder amenable to cell-based treatment comprising administering a pharmaceutically effective amount of a retinal pigment epithelial cell obtained by the method of claim 14.

25. A pharmaceutical composition comprising a cord blood monocyte derived stem cell and a pharmaceutically acceptable diluent, carrier or medium.
